(19) United States
(12) Patent Application Publication
(10) Pub. No.: US 2007/0224601 A1
(43) Pub. Date: Sep. 27, 2007

(54) Quantification of depurination through the use of two cleavage sites

(76) Inventors: Heather Brummell McCuen, San Jose, CA (US); Eric M. Leproust, San Jose, CA (US)

Correspondence Address: AGILENT TECHNOLOGIES INC., INTELLECTUAL PROPERTY ADMINISTRATION,LEGAL DEPT., MS BLDG. E P.O. BOX 7599, LOVELAND, CO, 80537, US

(21) Appl. No.: 11/388,727

(22) Filed: Mar. 24, 2006

(51) Int. Cl. C12Q 1/68

(52) U.S. Cl. 435/6

(57) ABSTRACT

The invention is directed an array comprising at least one depurination probe including a depurination sensitive first cleavage site adjacent a first tag region; and a base-cleavable second cleavage site adjacent a second tag region wherein the first tag region and the second tag region are independently quantifiable upon release from the depurination probe; and a method for use thereof to quantify depurination side reactions and overall yield during in situ array synthesis.

The present invention relates to biopolymeric arrays, particularly in situ produced nucleic acid arrays, and more particularly the quality assessment thereof.

### BACKGROUND

Biopolymer arrays can be fabricated using either deposition of the previously obtained biopolymers or in situ synthesis methods. Such in situ synthesis methods can be basically regarded as iterating the sequence of depositing droplets of: (a) a protected monomer onto predetermined locations on a substrate to link with either a suitably activated substrate surface (or with previously deposited deprotected monomer); (b) deprotecting the deposited monomer so that it can react with a subsequently deposited protected monomer; and (c) depositing another protected monomer for linking. Different monomers may be deposited at different regions on the substrate during any one cycle so that the different regions of the completed array will carry the different biopolymer sequences as desired in the completed array. One or more intermediate further steps may be required in each iteration, such as oxidation and washing steps.

The synthesis protocol used to fabricate an array of biopolymeric probes can have a significant impact on the functional nature of the in situ synthesized probes and features thereof on the array. For example, the particular probe synthesis protocol employed can have an impact on the percentage of full length probes that are produced in a given feature. In other words, a given in situ synthesis protocol may produce, in addition to full length probe sequences, non-full length sequences, which non-full length sequences can adversely impact the functionality of the feature.

One reason that non-full length sequences may be produced, in addition to desired full length sequences, in a given feature of an array is that in situ produced oligonucleotides are susceptible to depurination side reactions, specifically acid-catalyzed depurination, shown in below in Scheme 1.

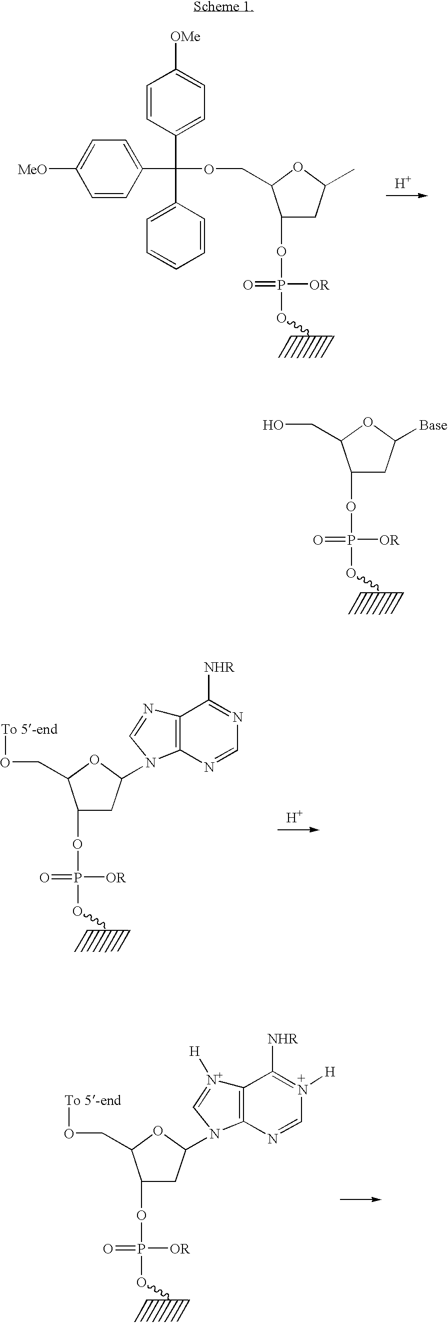

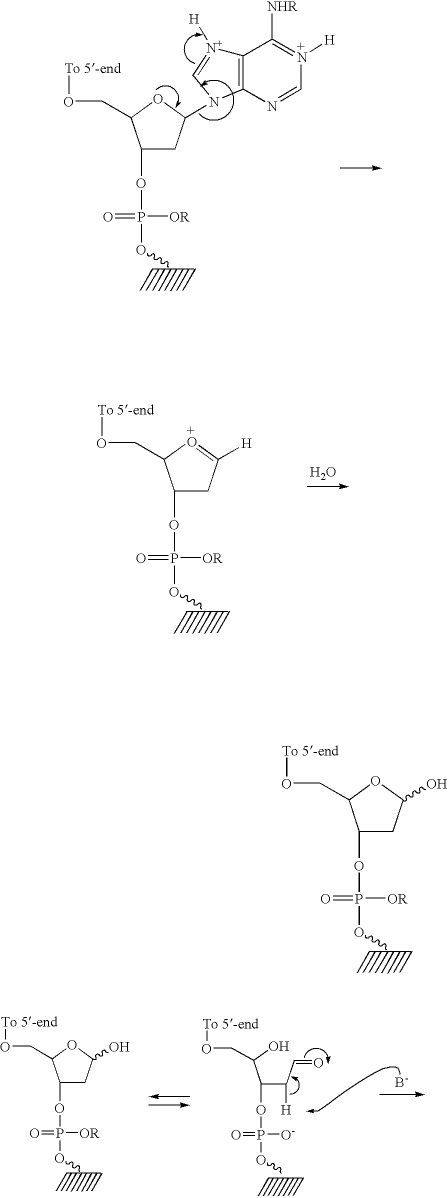

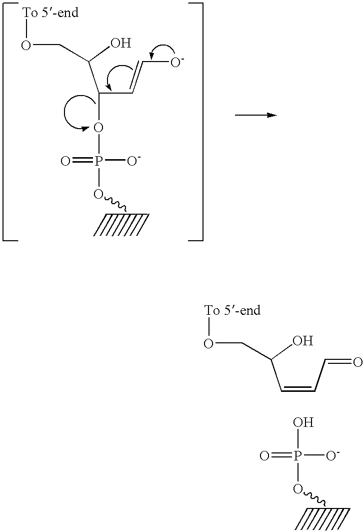

The first line of Scheme 1 shows the desired reaction (deblocking the 5′-hydroxyl at the end of each synthetic cycle) that is responsible for cyclic acid exposure. The second line shows the undesirable, acid-catalyzed side reaction: hydrolysis of the deoxyribose-purine (glycosidic) bond, with conversion of the furan structure of the deoxyribose sugar into an aldose. The base shown in Scheme 1 is adenine (A), because A is by far the more sensitive of the 2 purines. The final line of Scheme 1 shows the eventual consequences of depurination when the finished oligonucleotide is exposed to a final, base-catalyzed deprotection step to remove protecting groups from the A, C and G bases: the 3′-phosphodiester bond to the aldose sugar is cleaved by β-elimination, cleaving the oligonucleotide backbone, with loss of all bases on the 5′-side of the site of depurination.

Depurination of array-bound oligonucleotides is a particularly pernicious problem in those manufacturing protocols where the oligonucleotides on an in situ-synthesized microarray are not subjected to subsequent purification steps meant to retain only full-length products. Thus, depurination during a given synthesis protocol may yield a microarray feature that is both depleted in the intended, full-length oligonucleotide and filled with truncated sequences, where these non-full length sequences at best do nothing and at worst degrade the specificity of the full-length probes.

In view of above described potentially serious impact of depurination on array quality, the detection of depurination is an important component of the overall assessment of microarray quality. As such, there is a need for the development of methods to assess depurination during the in situ manufacture of a nucleic acid array.

### SUMMARY

Methods for detecting depurination reaction products from an in situ produced nucleic acid array are provided and arrays for use therein, where the nucleic acid array includes at least one depurination feature is made up of a first cleavage site adjacent a first tag region, wherein the first cleavage site undergoes base-sensitive cleavage if depurinated; and a second cleavage site adjacent a second tag region, wherein the second cleavage site is base-sensitive. In the present method, the arrays are contacted with a basic solution during a post-synthesis deprotection step. The amount of the first tag region released from the array due to cleavage at the first cleavage site is determined to evaluate the extent of depurination that occurred during in situ synthesis of the array. In additional embodiments, the amount of the second tag region released from the array due to cleavage of the second cleavage site is determined to evaluate overall synthetic yield on the array. The subject arrays find use in a variety of different applications, including array fabrication quality control applications, e.g., to determine the extent of depurination in a given lot of nucleic acid arrays produced using an in situ fabrication protocol.

### DESCRIPTION OF THE DRAWINGS

FIG. 1 shows an exemplary substrate carrying an array, such as may be used in the devices of the subject invention.

FIG. 2 shows an enlarged view of a portion of FIG. 1 showing spots or features.

FIG. 3 is an enlarged view of a portion of the substrate of FIG. 1.

FIG. 4 provides a depiction of a representative depurination probe according to an embodiment of the subject invention.

### DESCRIPTION—DEFINITIONS

Various embodiments of the present invention will be described in detail with reference to the drawings, wherein like reference numerals represent like parts throughout the several views. Reference to various embodiments does not limit the scope of the invention, which is limited only by the scope of the claims attached hereto. Additionally, any examples set forth in this specification are not intended to be limiting and merely set forth some of the many possible embodiments for the claimed invention.

In this specification and the appended claims, the singular forms “a,” “an” and “the” include plural reference unless the context clearly dictates otherwise.

Where a range of values is provided, it is understood that each intervening value, to the tenth of the unit of the lower limit unless the context clearly dictates otherwise, between the upper and lower limit of that range, and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

All patents and other references cited in this application, are incorporated into this application by reference except insofar as they may conflict with those of the present application (in which case the present application prevails).

Unless defined otherwise, all technical and scientific terms used herein have the same meaning as commonly understood by one of ordinary skill in the art to which this invention belongs. Still, certain elements are defined below for the sake of clarity and ease of reference.

The terms “nucleoside” and “nucleotide” are intended to include those moieties that contain not only the known purine and pyrimidine base moieties, but also other heterocyclic base moieties that have been modified. Such modifications include methylated purines or pyrimidines, acylated purines or pyrimidines, or other heterocycles. In addition, the terms “nucleoside” and “nucleotide” include those moieties that contain not only conventional ribose and deoxyribose sugars, but other sugars as well. Modified nucleosides or nucleotides also include modifications on the sugar moiety, e.g., wherein one or more of the hydroxyl groups are replaced with halogen atoms or aliphatic groups, or are functionalized as ethers, amines, or the like.

The term “oligonucleotide” as used herein denotes single stranded nucleotide multimers of from about 10 to 100 nucleotides and up to 200 nucleotides in length.

A “biopolymer” is a polymer of one or more types of repeating units. Biopolymers are typically found in biological systems and particularly include polysaccharides (such as carbohydrates), peptides (which term is used to include polypeptides and proteins) and polynucleotides as well as their analogs such as those compounds composed of or containing amino acid analogs or non-amino acid groups, or nucleotide analogs or non-nucleotide groups. Biopolymers include polynucleotides in which the conventional backbone has been replaced with a non-naturally occurring or synthetic backbone, and nucleic acids (or synthetic or naturally occurring analogs) in which one or more of the conventional bases has been replaced with a group (natural or synthetic) capable of participating in Watson-Crick type hydrogen bonding interactions. Polynucleotides include single or multiple stranded configurations, where one or more of the strands may or may not be completely aligned with another. A “nucleotide”refers to a sub-unit of a nucleic acid and has a phosphate group, a 5 carbon sugar and a nitrogen containing base, as well as functional analogs (whether synthetic or naturally occurring) of such sub-units which in the polymer form (as a polynucleotide) can hybridize with naturally occurring polynucleotides in a sequence specific manner analogous to that of two naturally occurring polynucleotides. Biopolymers include DNA (including cDNA), RNA, oligonucleotides, and PNA and other polynucleotides as described in U.S. Pat. No. 5,948,902 and references cited therein (all of which are also incorporated herein by reference), regardless of the source. An “oligonucleotide” generally refers to a nucleotide multimer of about 10 to 100 nucleotides in length, while a “polynucleotide” includes a nucleotide multimer having any number of nucleotides. A “biomonomer” references a single unit, which can be linked with the same or other biomonomers to form a biopolymer (e.g., a single amino acid or nucleotide with two linking groups one or both of which may have removable protecting groups).

An “array,” includes any one-dimensional, two-dimensional or substantially two-dimensional (as well as a three-dimensional) arrangement of addressable regions bearing a particular chemical moiety or moieties (e.g., biopolymers such as polynucleotide or oligonucleotide sequences (nucleic acids), polypeptides (e.g., proteins), carbohydrates, lipids, etc.) associated with that region. In the broadest sense, the preferred arrays are arrays of polymeric binding agents, where the polymeric binding agents may be any of: polypeptides, proteins, nucleic acids, polysaccharides, synthetic mimetics of such biopolymeric binding agents, etc. In many embodiments of interest, the arrays are arrays of nucleic acids, including oligonucleotides, polynucleotides, cDNAs, mRNAs, synthetic mimetics thereof, and the like. Where the arrays are arrays of nucleic acids, the nucleic acids may be covalently attached to the arrays at any point along the nucleic acid chain, but are generally attached at one of their termini (e.g. the 3′ or 5′ terminus). Sometimes, the arrays are arrays of polypeptides, e.g., proteins or fragments thereof.

Any given substrate may carry one, two, four or more or more arrays disposed on a front surface of the substrate. Depending upon the use, any or all of the arrays may be the same or different from one another and each may contain multiple spots or features. A typical array may contain more than ten, more than one hundred, more than one thousand more ten thousand features, or even more than one hundred thousand features, in an area of less than 20 cm² or even less than 10 cm². For example, features may have widths (that is, diameter, for a round spot) in the range from a 10 μm to 1.0 cm. In other embodiments each feature may have a width in the range of 1.0 μm to 1.0 mm, usually 5.0 μm to 500 μm, and more usually 10 μm to 200 μm. Non-round features may have area ranges equivalent to that of circular features with the foregoing width (diameter) ranges. At least some, or all, of the features are of different compositions (for example, when any repeats of each feature composition are excluded the remaining features may account for at least 5%, 10%, or 20% of the total number of features). Interfeature areas will typically (but not essentially) be present which do not carry any polynucleotide (or other biopolymer or chemical moiety of a type of which the features are composed). Such interfeature areas typically will be present where the arrays are formed by processes involving drop deposition of reagents but may not be present when, for example, light directed synthesis fabrication processes are used. It will be appreciated though, that the interfeature areas, when present, could be of various sizes and configurations.

Each array may cover an area of less than 100 cm², or even less than 50 cm², 10 cm² or 1 cm². In many embodiments, the substrate carrying the one or more arrays will be shaped generally as a rectangular solid (although other shapes are possible), having a length of more than 4 mm and less than 1 m, usually more than 4 mm and less than 600 mm, more usually less than 400 mm; a width of more than 4 mm and less than 1 m, usually less than 500 mm and more usually less than 400 mm; and a thickness of more than 0.01 mm and less than 5.0 mm, usually more than 0.1 mm and less than 2 mm and more usually more than 0.2 and less than 1 mm. With arrays that are read by detecting fluorescence, the substrate may be of a material that emits low fluorescence upon illumination with the excitation light. Additionally in this situation, the substrate may be relatively transparent to reduce the absorption of the incident illuminating laser light and subsequent heating if the focused laser beam travels too slowly over a region. For example, substrate 10 may transmit at least 20%, or 50% (or even at least 70%, 90%, or 95%), of the illuminating light incident on the front as may be measured across the entire integrated spectrum of such illuminating light or alternatively at 532 nm or 633 nm.

Arrays can be fabricated using drop deposition from pulsejets of either polynucleotide precursor units (such as monomers) in the case of in situ fabrication, or the previously obtained polynucleotide. Such methods are described in detail in, for example, the previously cited references including U.S. Pat. No. 6,242,266, U.S. Pat. No. 6,232,072, U.S. Pat. No. 6,180,351, U.S. Pat. No. 6,171,797, U.S. Pat. No. 6,323,043, U.S. patent application Ser. No. 09/302,898 filed Apr. 30, 1999 by Caren et al., and the references cited therein. These references are incorporated herein by reference. Other drop deposition methods can be used for fabrication, as previously described herein. Also, instead of drop deposition methods, light directed fabrication methods may be used, as are known in the art. Interfeature areas need not be present particularly when the arrays are made by light directed synthesis protocols.

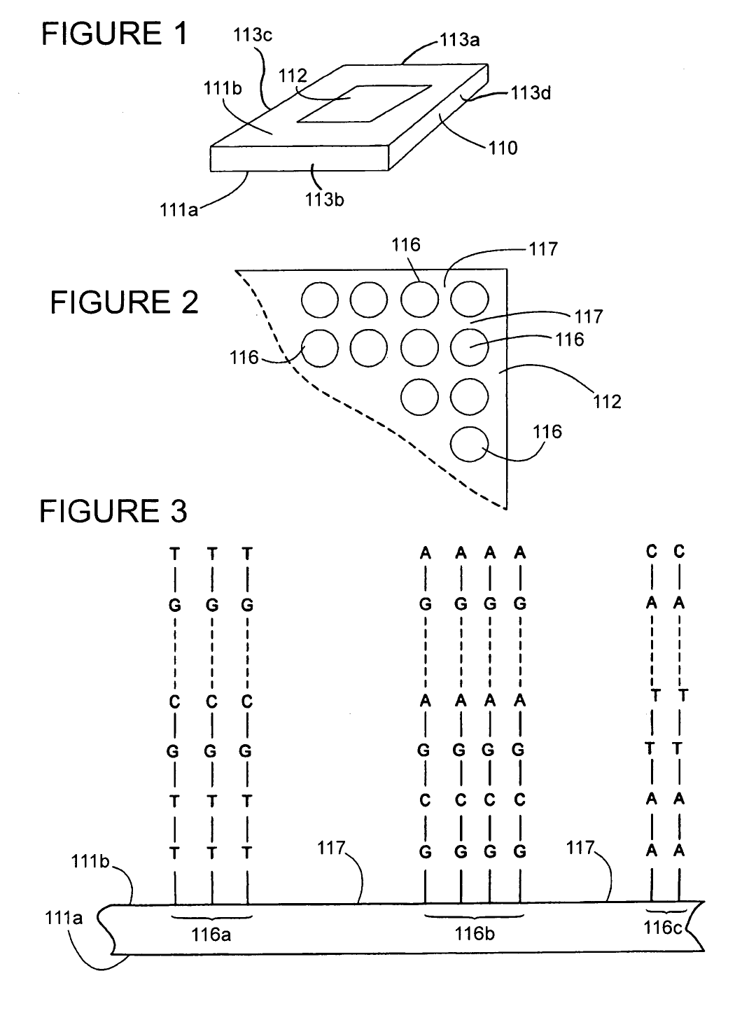

An exemplary array is shown in FIGS. 1-3, where the array shown in this representative embodiment includes a contiguous planar substrate 110 carrying an array 112 disposed on a rear surface 111b of substrate 110. It will be appreciated though, that more than one array (any of which are the same or different) may be present on rear surface 111b, with or without spacing between such arrays. That is, any given substrate may carry one, two, four or more arrays disposed on a front surface of the substrate and depending on the use of the array, any or all of the arrays may be the same or different from one another and each may contain multiple spots or features. The one or more arrays 112 usually cover only a portion of the rear surface 111b, with regions of the rear surface 111b adjacent the opposed sides 113c, 113d and leading end 113a and trailing end 113b of slide 110, not being covered by any array 112. A front surface 111a of the slide 110 does not carry any arrays 112. Each array 112 can be designed for testing against any type of sample, whether a trial sample, reference sample, a combination of them, or a known mixture of biopolymers such as polynucleotides. Substrate 110 may be of any shape, as mentioned above.

As mentioned above, array 112 contains multiple spots or features 116 of biopolymers, e.g., in the form of polynucleotides. As mentioned above, all of the features 116 may be different, or some or all could be the same. The interfeature areas 117 could be of various sizes and configurations. Each feature carries a predetermined biopolymer such as a predetermined polynucleotide (which includes the possibility of mixtures of polynucleotides). It will be understood that there may be a linker molecule (not shown) of any known types between the rear surface 111b and the first nucleotide.

Substrate 110 may carry on front surface 111a, an identification code, e.g., in the form of bar code (not shown) or the like printed on a substrate in the form of a paper label attached by adhesive or any convenient means. The identification code contains information relating to array 112, where such information may include, but is not limited to, an identification of array 112, i.e., layout information relating to the array(s), etc.

An array is “addressable” when it has multiple regions of different moieties (e.g., different polynucleotide sequences) such that a region (i.e., a “feature” or “spot” of the array) at a particular predetermined location (i.e., an “address”) on the array will detect a particular target or class of targets (although a feature may incidentally detect non-targets of that feature). Array features are typically, but need not be, separated by intervening spaces. In the case of an array, the “target” will be referenced as a moiety in a mobile phase (typically fluid), to be detected by probes (“target probes”) which are bound to the substrate at the various regions. However, either of the “target” or “target probe” may be the one which is to be evaluated by the other (thus, either one could be an unknown mixture of polynucleotides to be evaluated by binding with the other). A “scan region” refers to a contiguous (preferably, rectangular) area in which the array spots or features of interest, as defined above, are found. The scan region is that portion of the total area illuminated from which the resulting fluorescence is detected and recorded. For the purposes of this invention, the scan region includes the entire area of the slide scanned in each pass of the lens, between the first feature of interest, and the last feature of interest, even if there exist intervening areas which lack features of interest. An “array layout” refers to one or more characteristics of the features, such as feature positioning on the substrate, one or more feature dimensions, and an indication of a moiety at a given location. “Hybridizing” and “binding”, with respect to polynucleotides, are used interchangeably.

The term “substrate” as used herein refers to a surface upon which marker molecules or probes, e.g., an array, may be adhered. Glass slides are the most common substrate for biochips, although fused silica, silicon, plastic and other materials are also suitable.

### Description—Arrays Including Depurination Probes

In further describing the invention in greater detail than provided in the Summary and as informed by the Background and Definitions provided above, representative embodiments and portions of the depurination probes and subject arrays are described first in greater detail, followed by a review of representative applications of such arrays, e.g., in quality assessment.

In situ produced nucleic acid arrays that include at least one depurination probe feature are provided, where the depurination probe feature includes at least one depurination probe including a purine cleavage site adjacent a first tag region and a second cleavage site adjacent a second tag region, wherein the second cleavage site is base-sensitive. The purine cleavage site becomes base-sensitive only when previously depurinated, for example during array synthesis.

In producing the subject arrays, the depurination probe features are synthesized in situ concurrently with other array features or an array containing depurination probe features is synthesized concurrently with other arrays on a wafer. The final step of in situ array synthesis protocols is to contact the array or arrays on a wafer surface with a basic solution to deprotect the synthesized probes. During treatment with the basic solution, the base-sensitive second cleavage site, and any depurinated purine cleavage sites are cleaved. Following contact with the basic solution, the released tag regions are collected from the array, and are detected and/or quantitated by any of a number of known techniques including but not limited to mass spectroscopy, HPLC, and gel electrophoresis.

The amount of the first tag region released from the array is related to depurination because the purine cleavage site adjacent the first tag region is cleaved in the basic deprotection step only when previously depurinated, typically during array synthesis. Therefore evaluation of the first tag region released is used to determine the extent of depurination that occurred during in situ synthesis of the array. In additional embodiments, the amount of the second tag region released from the array due to cleavage of the base-sensitive second cleavage site is determined to evaluate overall synthetic yield on the array. The subject arrays find use in a variety of different applications, including array fabrication quality control applications, e.g., to determine the extent of depurination in a given lot of nucleic acid arrays produced using an in situ fabrication protocol.

Depurination Probe

The subject invention provides nucleic acid arrays that include at least one depurination probe feature. Each depurination probe feature of the subject arrays is made up of one or more depurination probes produced in situ on an array with the other array features. The depurination probes are generally made with the same materials and processes as other features applied in situ on an array surface; for example, nucleic acids or derivatives thereof for a nucleic acid array. Generally, a depurination probe is a linear molecule attached at one terminus, either directly or indirectly, covalently or non-covalently, to an array surface.

Each depurination probe includes two cleavage sites. A first cleavage site or purine cleavage site is a depurination sensitive site. If the first cleavage site is depurinated during synthesis of the array probes, the site becomes base-sensitive, i.e. cleaves upon contact with basic solution. Cleavage at the first cleavage site provides information (e.g., qualitative or quantitative) regarding depurination on the array surface during in situ synthesis. The second cleavage site is a base sensitive cleavage site. Cleavage at the second cleavage site provides quantification of synthetic yield on the array and detection of coupling failure.

In an embodiment, each depurination probe includes: a first cleavage site; a first tag region; a second cleavage site; and a second tag region. The first cleavage site is adjacent a first tag region such that cleavage of the first cleavage site releases at least the first tag region from the array surface. The second cleavage site is adjacent a second tag region such that cleavage of the second cleavage site releases the second tag region from the array surface. The first cleavage site with first tag region and second cleavage site with second tag region are typically part of linear probe wherein the order from near the array surface to farthest from the array surface is first cleavage site, first tag region, second cleavage site, second tag region. In an embodiment, the depurination probe is a nucleic acid molecule, such as: 3′-first cleavage site—first tag domain—second cleavage site—second tag domain-5′. In a further embodiment, the nucleic acid depurination probe includes 3′-AA-[first tag region]-[base-sensitive cleavage site]-[second tag region]-5′. In various embodiments a depurination probe may include additional components, such as but not limited to spacers for association of the probe to the array surfaces as well as other regions and/or functional moieties.

The first cleavage site includes a purine cleavage site. The purine cleavage site has one or more purine bases, preferably adenosine. In an embodiment, each purine cleavage site has one to six purine bases. In a further embodiment each depurination probe feature has one to three purine bases. In still further embodiments, the purine cleavage site is two adenosine bases. The purine cleavage site is proximal to the array surface and adjacent the first tag region. In an embodiment, the purine cleavage site is located at or near the 3′ end of the depurination probe where the 3′ end of the depurination probe is attached to the array surface.

The second cleavage site includes a base-cleavable site. Base-cleavable sites are base-sensitive, i.e. cleaves upon treatment with basic solution under conditions wherein other the majority of other array probes are not subject to cleavage. In an embodiment, the second cleavage site includes at least one chemically modified nucleic acid which is base-sensitive. Examples of chemically modifications include, but are not limited to, ester groups, sulfone groups, and succinates incorporated into the nucleic acid backbone of the depurination probe at the second cleavage site. See also, for example, U.S. Pat. Appl. Pub. 2002/0009729. In a particular embodiment, the second cleavage site includes a thymidine-succinyl hexamide CED phosphoramidite linker available from ChemGenes Corporation of Wilmington, Mass.

The second tag region is located at the end of the depurination probe most distant from the surface upon which the probe is immobilized. The second cleavage site connects the second tag region with the remainder of the depurination probe. In an embodiment, a second tag region is preferentially formed of pyrimidine residues.

The combination of the second cleavable site with the second tag region may also be referred to as a base-cleavable linker. One example of a suitable second cleavage site adjacent a second tag region is one chemically modified nucleic acid which is base-sensitive adjacent approximately 25 pyrimidine nucleotides. In a further embodiment, the base-sensitive modified nucleic acid feature links the 25 pyrimidine nucleotides with the remainder of the array-associated depurination probe. In particular embodiments, a suitable second cleavage site adjacent a second tag region or base-cleavable linker are referred to as a cleavable T-linker or C-linkers.

The first tag region and the second tag region are generally formed from nucleic acids. Preferably, the tag regions do not include base-sensitive nucleic acids or nucleic acids highly susceptible to depurination, such as adenosine residues, to avoid errors in measurement due to depurination within the tag region. In further embodiments, the tag regions are formed of pyrimidines. In an embodiment, each tag region is homogenous with respect to the residues which it includes. In an embodiment, one or both of the tag regions are a poly-T region.

Each tag region independently ranges in length from about 1 to 70 nucleotides, including from about 1 to 35 nucleotides. Preferably, the length in nucleotides of the first tag region and the second tag region are different. In an embodiment, the first tag region and second tag region are differentially detectable by analysis techniques such as, but not limited to HPLC, mass spectroscopy, and/or gel electrophoresis. In a further embodiment, the difference in size between the first tag region and second tag region is four or more bases or nucleotides (nt) in length.

An embodiment of a full length depurination probe of the depurination probe feature(s) of the subject arrays ranges in length from about 5 to about 100, such as from about 10 to about 80, including from about 25 to about 60. In an embodiment, the length of the depurination probes is matched to the length of the nucleic acid probes present in other features on the array. The size of one or more of the cleavage sites and tag regions may be adjusted as needed for different depurination probe lengths.

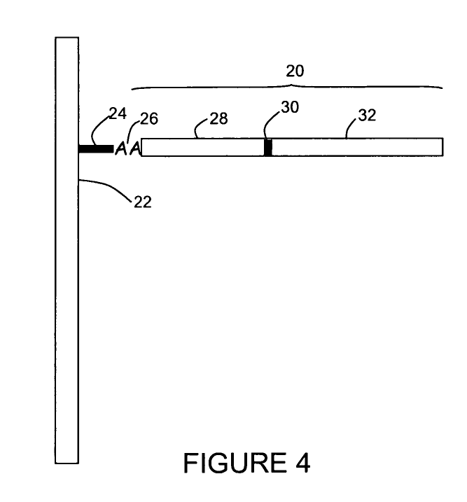

One specific example embodiment of a depurination probe feature on an array is shown in FIG. 4. The depurination probe 20 is attached to array surface 22 at its 3′ end by a spacer 24. Depurination probe 20, described from 3′ to 5′, includes first cleavage site 26 including two A's, adjacent to first tag region 28 of twenty-five T's, followed by second cleavage site 30, adjacent to second tag region 32 of twenty T's.

As a representative example, depurination probe, such as depurination probe 20 may be designed having 60 nucleotides in length for an array of 60-mer probes. For example, the first tag region is 25 nt, and the second tag region is 20 nt. The second cleavage site is one chemically modified nucleic acid which is base-sensitive, while the first cleavage site is A-A, which is only base-sensitive if previously depurinated, for example during previous synthesis steps. The remaining 10 residues, preferably pyrimidines to avoid depurination, are attached between the spacer on the array surface and the first cleavage site.

Arrays Containing Depurination Features

The subject invention provides nucleic acid arrays that include at least one depurination probe feature. In an embodiment, an array contains at least one spot having a plurality of depurination probes. In a further embodiment, an array includes a plurality of spots containing a plurality of depurination probes. In a still further embodiment, an array contains only depurination probes.

Arrays containing depurination probe features or arrays formed concurrently with arrays containing depurination probe features on a common wafer surface also typically include at least two distinct nucleic acids that differ by monomeric sequence immobilized on e.g., covalently or non-covalently attached to, different and known locations on the substrate surface. Each distinct nucleic acid sequence of the array is typically present as a composition of multiple copies of the polymer on the substrate surface, e.g., as a spot on the surface of the substrate. The number of distinct nucleic acid sequences, and hence spots or similar structures (i.e., array features), present on the array may vary, but is generally at least 2, usually at least about 5 and more usually at least about 10, where the number of different spots on the array may be as a high as about 50, about 100, about 500, about 1000, about 10,000 or higher, depending on the intended use of the array. The spots of distinct nucleic acids present on the array surface are generally present as a pattern, where the pattern may be in the form of organized rows and columns of spots, e.g., a grid of spots, across the substrate surface, a series of curvilinear rows across the substrate surface, e.g., a series of concentric circles or semi-circles of spots, and the like. The density of spots present on the array surface may vary, but will generally be at least about 10 and usually at least about 100 spots/cm², where the density may be as high as 10⁶ or higher, but will generally not exceed about 10⁵ spots/cm². The total amount of nucleic acid in a given feature may range from about 1×10⁻⁴ pmol to about 0.1 pmol, such as from about 1×10⁻³ pmol to about 1×10⁻³ pmol.

In the subject arrays of nucleic acids, the nucleic acids, including depurination probe features and other nucleic acid features, may be covalently attached to the arrays at any point along the nucleic acid chain, but are generally attached at one of their termini, e.g., the 3′ or 5′ terminus. In many embodiments, each nucleic acid feature, including the depurination features described herein is attached to the array surface at their 3′ terminus.

A feature of the subject arrays is that they include at least one depurination feature. The number of depurination features may vary, but is in certain embodiments less than about 300, such as less than about 100 and including less than about 70, where the number may be as high as 600 or higher in certain embodiments, but in many embodiments does not exceed about 70. The features or spots of the subject arrays containing depurination probes described above can be positioned at any location on the array. For example, the depurination probes can be positioned in different areas, addressable regions, sections, rows or columns of an array or wafer containing one or more arrays, as convenient. In an embodiment, a plurality of arrays are concurrently formed in situ on a wafer wherein one of the plurality of arrays contains features including depurination probes. In a further embodiment, one array of a plurality of arrays formed concurrently in situ on a wafer includes only depurination probes, while the other arrays of the plurality contain other features. In a still further embodiment, the array containing the depurination probe features is formed concurrently in situ on a wafer with a plurality of arrays, and after synthesis, the array containing depurination probe features is separated from the wafer and processed to determine depurination and yield of the array synthesis process on the wafer.

In Situ Synthesis of Arrays Containing Depurination Probe Features

The depurination probe features described herein are generally synthesized in situ with other nucleotide array features. The synthesis of arrays of polynucleotide probes on the surface of a support in certain approaches, e.g., in situ fabrication protocols, involves attaching an initial nucleoside or nucleotide to a functionalized surface. In one approach the surface is reacted with nucleosides or nucleotides that are also functionalized for reaction with the groups on the surface of the support. Methods for introducing appropriate amine specific or alcohol specific reactive functional groups into a nucleoside or nucleotide include, by way of example, addition of a spacer amine containing phosphoramidites, addition on the base of alkynes or alkenes using palladium mediated coupling, addition of spacer amine containing activated carbonyl esters, addition of boron conjugates, formation of Schiff bases.

After the introduction of the nucleoside or nucleotide onto the surface, the attached nucleotide may be used to construct the polynucleotide by means well known in the art. For example, in the synthesis of arrays of oligonucleotides, nucleoside monomers are generally employed. In this embodiment an array of the above compounds is attached to the surface and each compound is reacted to attach a nucleoside. Nucleoside monomers are used to form the polynucleotides usually by phosphate coupling, either direct phosphate coupling or coupling using a phosphate precursor such as a phosphite coupling. Such coupling thus includes the use of amidite (phosphoramidite), phosphodiester, phosphotriester, H-phosphonate, phosphite halide, and the like coupling.

One exemplary coupling method is phosphoramidite coupling, which is a phosphite coupling. In using this coupling method, after the phosphite coupling is complete, the resulting phosphite is oxidized to a phosphate. Oxidation can be effected with iodine to give phosphates or with sulfur to give phosphorothioates. The phosphoramidites are dissolved in a suitable organic solvent or solvent mixture to give a solution having a given ratio of amidite concentrations. The mixture of known chemically compatible monomers is reacted to a solid support, or further along, may be reacted to a growing chain of monomer units. In one particular example, the terminal 5′-hydroxyl group is caused to react with a deoxyribonucleoside-3′-O—(N,N-diisopropylamino)phosphoramidite protected at the 5′-position with dimethoxytrityl or the like. The 5′ protecting group is removed after the coupling reaction, and the procedure is repeated with additional protected nucleotides until synthesis of the desired polynucleotide is complete. For a more detailed discussion of the chemistry involved in the above synthetic approaches, see, for example, U.S. Pat. No. 5,436,327 at column 2, line 34, to column 4, line 36, which is incorporated herein by reference in its entirety.

In general, in the above synthetic steps involving monomer addition such as, for example, the phosphoramidite method, there are certain repetitive steps such as washing the surface of the support prior to or after a reaction, oxidation of substances such as oxidation of a phosphite group to a phosphate group, removal of protecting groups, blocking of sites to prevent reaction at such site, capping of sites that did not react with a phosphoramidite reagent, deblocking, and so forth. In addition, under certain circumstances other reactions may be carried out in a flow cell such as, for example, phosphoramidite monomer addition, modified phosphoramidite addition, other monomer additions, addition of a polymer chain to a surface for linking to monomers, and so forth.

For in situ fabrication methods, multiple different reagent droplets are deposited by pulse jet or other means at a given target location in order to form the final feature (hence a probe of the feature is synthesized on the array substrate). The in situ fabrication methods include those described in U.S. Pat. No. 5,449,754 for synthesizing peptide arrays, and in U.S. Pat. No. 6,180,351 and WO 98/41531 and the references cited therein for polynucleotides, and may also use pulse jets for depositing reagents. The in situ method for fabricating a polynucleotide array typically follows, at each of the multiple different addresses at which features are to be formed, the same conventional iterative sequence used in forming polynucleotides from nucleoside reagents on a support by means of known chemistry. This iterative sequence can be considered as multiple ones of the following attachment cycle at each feature to be formed: (a) coupling an activated selected nucleoside (a monomeric unit) through a phosphite linkage to a functionalized support in the first iteration, or a nucleoside bound to the substrate (i.e. the nucleoside-modified substrate) in subsequent iterations; (b) optionally, blocking unreacted hydroxyl groups on the substrate bound nucleoside (sometimes referenced as “capping”); (c) oxidizing the phosphite linkage of step (a) to form a phosphate linkage; and (d) removing the protecting group (“deprotection”) from the now substrate bound nucleoside coupled in step (a), to generate a reactive site for the next cycle of these steps. The coupling can be performed by depositing drops of an activator and phosphoramidite at the specific desired feature locations for the array. The functionalized support (in the first cycle) or deprotected coupled nucleoside (in subsequent cycles) provides a substrate bound moiety with a linking group for forming the phosphite linkage with a next nucleoside to be coupled in step (a). Capping, oxidation and deprotection can be accomplished by treating the entire substrate (“flooding”) with a layer of the appropriate reagent. The in situ nucleic acid synthesis protocol generally includes a plurality of cycles, as described above, each cycle including: printing (e.g., coupling the activated nucleoside to the substrate), oxidizing, and deblocking (e.g., preparing the nucleoside for the next coupling reaction by generating 5′ functional group). Upon completion of multiple cycles of nucleotide addition, for example 60 rounds for a 60-mer probe, a final deprotection of nucleoside bases is performed under basic conditions. Final deprotection of nucleoside bases can be accomplished using basic (i.e. alkaline) conditions, by a flooding procedure in a known manner. Basic conditions refers to treatment of the entire substrate surface, including one or more newly printed arrays with an alkaline solution.

For depurination probes described herein, suitable basic conditions cause cleavage of depurinated first cleavage sites and cleavage of the majority, preferably all of the second cleavage sites upon treatment for a period of time with the basic solution. Preferably, other nucleic acid probes, e.g., undamaged full length probes on the array are deprotected as needed, but not otherwise damaged by the basic conditions. In particular, first cleavage sites that were not depurinated are not cleaved by treatment under basic conditions.

In general, the basic solution must have sufficient basicity to bring about cleavage of depurinated first domains and cleavage of base-sensitive second domains. The base should have a conjugate acid of pKa of about 9 to 12 and should not cleave the attachment linkage between the DNA oligonucleotide and the glass surface. Suitable alkaline or basic solutions include, but are not limited to aqueous or alcoholic solutions including a base, such as but not limited to ammonium hydroxide, alkyldiamines, ammonia, or the like. The basic solution may include, for example, ethanolamine, ammonia, mixtures of an alkyl diamine such as, e.g., ethylene diamine, methyl amine, etc., with a lower alkyl alcohol such as, e.g., ethanol, and the like. Specific examples, by way of illustration and not limitation, include 1:1 ethylene diamine:ethanol, 1:1 methyl amine:ethanol, and so forth. Other cleavage reagents will be apparent to one skilled in the art in view of the disclosure hereinabove. In an embodiment, one example of basic conditions is 50% ethylenediamine in ethanol for 1-2 hours at room temperature. In another embodiment, the array was dried prior to deprotection and reacted with ethanolamine for 30 min at room temperature to achieve both cleavage of any depurinated depurination cleavage sites and the base-cleavable linkers of the depurination probes. Additionally, base protecting groups may be removed. After rinsing in deionized water and drying, the array may be diced into 1 by 3 inch slides. Strength of the basic solution, temperature and time of treatment are adjusted to achieve the desired result.

Following treatment with basic solution, the support is treated to remove unbound reagents (cleaved second domains and first domains from its surface. To this end, the basic solution is collected. In addition, the support may be subjected to one or more wash steps. Wash steps can be carried out in a flow chamber or the support may be removed from the flow chamber and washed in a different location such as a wash station. The primary concern in washing the surface of the support is to remove and collect unbound materials from the depurination probe features for analysis.

The wash reagent is a protic solvent or an aprotic solvent suitable for solvating unbound second and/or first tag regions. Accordingly, the solvent may be an organic solvent such as, by way of illustration and not limitation, oxygenated organic solvents of from 1 to about 6, more usually from 1 to about 4, carbon atoms, including alcohols such as methanol, ethanol, propanol, etc., ethers such as tetrahydrofuran, ethyl ether, propyl ether, etc., acetonitrile, dimethylformamide, dimethylsulfoxide, dichloromethane, toluene, and the like. Alternatively, the solvent may be an aqueous medium that is solely water or may contain a buffer, or may contain from about 0.01 to about 80 or more volume percent of a cosolvent such as an organic solvent as mentioned above.

The foregoing chemistry of the synthesis of polynucleotides is described in detail, for example, in Caruthers, Science 230: 281-285, 1985; Itakura, et al., Ann. Rev. Biochem. 53: 323-356; Hunkapillar, et al., Nature 310: 105-110, 1984; and in “Synthesis of Oligonucleotide Derivatives in Design and Targeted Reaction of Oligonucleotide Derivatives”, CRC Press, Boca Raton, Fla., pages 100 et seq., U.S. Pat. Nos. 4,458,066, 4,500,707, 5,153,319, 5,869,643 and European patent application, EP 0294196, and elsewhere. The phosphoramidite and phosphite triester approaches are most broadly used, but other approaches include the phosphodiester approach, the phosphotriester approach and the H-phosphonate approach. The substrates are typically functionalized to bond to the first deposited monomer. Suitable techniques for functionalizing substrates with such linking moieties are described, for example, in Southern, E. M., Maskos, U. and Elder, J. K., Genomics, 13, 1007-1017, 1992.

In the case of array fabrication, different monomers and activator may be deposited at different addresses on the substrate during any one cycle so that the different features of the completed array will have different desired biopolymer sequences. One or more intermediate further steps may be required in each cycle, such as the conventional oxidation, capping and washing steps in the case of in situ fabrication of polynucleotide arrays (again, these steps may be performed in flooding procedure). This allows synthesis of depurination probes along with other biopolymer probe sequences on the array surface at the same time, thereby experiencing the same oxidizing, capping, deblocking, and finally base deprotection. By experiencing the same reaction conditions as the other biopolymer probe sequences, the depurination probes provide useful indications of depurination and yield across the support, which includes one or more arrays.

The 5′ functional group generating step (that includes oxidizing, optionally capping and deblocking (described in greater detail below)) may occur in a flow cell. As summarized above, the flow cell allows fluids to be passed through the chamber where the support is disposed. The support is mounted in the chamber in or on a holder. The housing usually further comprises at least one fluid inlet and at least one fluid outlet for flowing fluids into and through the chamber in which the support is mounted. In one approach, the fluid outlet may be used to vent the interior of the reaction chamber for introduction and removal of fluid by means of the inlet. On the other hand, fluids may be introduced into the reaction chamber by means of the inlet with the outlet serving as a vent and fluids may be removed from the reaction chamber by means of the outlet with the inlet serving as a vent.

In certain embodiments, the support may be one on which a single array including at least one depurination probe feature is synthesized. Alternatively, multiple arrays of chemical compounds may be synthesized on the support, which is then diced when the arrays are complete, i.e., cut, into single array supports. In this alternative approach the support, may have depurination probe features apportioned through the different array locations, or alternatively, may group the depurination probe features into a single array which is separated from the other arrays.

Utility

In addition to their utility as nucleic acid arrays, reviewed in greater detail below, the subject depurination probe containing arrays find use evaluating or determining, e.g., measuring or quantifying, the extent of depurination in a given in situ array fabrication protocol. In other words, the subject arrays find use in methods of determining the extent of depurination that occurred during a given in situ array synthesis protocol, such as an in situ synthesis manufacturing run.

In these embodiments, following manufacture of an array by an in situ synthesis protocol, the array is contacted with a basic solution to deprotect the synthesized probes. When an array surface having one or more depurination probes as described above, depurination can be detected and quantified. In addition, the depurination probes and/or features can be used to quantify yield and assess quality of the in situ synthesis. Following deprotection by contact with the array of a basic solution, the nucleic acids released from the array, e.g., second tag region and/or first tag region are collected, for example by collecting the basic deprotection solution and/or subsequent washes of the array surface. The nucleic acids released from the array surface into solution may be detected and quantitated (either relative amount or quantitative amount) by any of a number of known techniques including but not limited to mass spectroscopy, HPLC, and gel electrophoresis.

The presence (and amount) of the second tag region supplies a quantitative measure of the synthesis yields. Cleavage of the second cleavage site occurs in the presence of basic solution. Therefore, treatment of the depurination probe of FIG. 4, will generate a T-20 DNA probe. The yield of the T-20 DNA probe directly correlates with the number of depurination probes and/or depurination probe features produced on an array and/or wafer surface. Analysis of the second tag region, e.g., the T-20 DNA probe of FIG. 4, will provide a quantitative measure of the synthesis yields. Reduction in yield of the second tag region, e.g., the T-20 DNA probe of FIG. 4, is an indication of coupling problems during in situ synthesis. Advantageously, depurination, if present, does not damage either the T-20 probe or the T-linker. In an embodiment, the expected yield of the second tag region may be used as value or constant to determine actual yield.

Depurination during in situ synthesis is revealed if first tag region(s) are released from the array surface during basic deprotection. Depurination at the first cleavage site, e.g., the two A bases shown in FIG. 4, during in situ synthesis causes the purine cleavage site to become base-sensitive. Therefore, depurination at the first cleavage site of the probe of FIG. 4, if it occurs, will produce a T-25 probe when treated with basic solution as described herein. The amount of the first tag region released from the array is then employed to estimate the amount of depurination reaction products, i.e., depurinated probes, present in the array. The detection of the first tag region may be either qualitative or quantitative. The amount of the first tag region released from an array surface is directly proportional to the amount of depurination probes on the array that underwent depurination during synthesis. If little or no depurination of the depurination probe(s) has occurred during synthesis, the quantity of first tag region, e.g., the T-25 probe, released from the array surface will be low. If depurination of the depurination probes during synthesis is high, the quantity of the first tag region released from the array surface will be high because the probe will have been cleaved from the array at the depurinated A bases in the presence of the basic solution.

Following treatment of an array or substrate with basic solution, the amount of first tag region released from the array, if any, is analyzed. For example, the amount of first tag region nucleic acids cleaved from the depurination probe is proportional to the amount depurinated probes. Generally it is known or predictable how many full length depurination probes should be present on an array if no depurination reactions have occurred during synthesis. Therefore, the resultant analysis of quantity of the first tag region from the depurination probe features of an array may then be employed to make an evaluation or determination of the extent of depurination that occurred during in situ fabrication of the array. This evaluation may also be applied to a substrate upon which multiple arrays are synthesized, wherein at least one array includes one or more depurination probe features. The particular protocol employed to determine the magnitude of depurination from the input signal data may vary, e.g., depending on the nature of the depurination probes, the nature of the in situ protocol used to prepare the array, and the detection method used, etc.

The amount of the second tag region released from the array is used to make a determination of quality and/or quantity of full length probes synthesized. Since the second tag region is released by cleavage of the base-sensitive cleavage site, the amount of second tag region detected should be directly proportional to the number of full length depurination probes and/or features synthesized. This yield of depurination probes synthesized, as indicated by second tag regions released, can then be used to predict the amount of full length probes synthesized in a feature and/or an array. This evaluation may also be applied to a substrate upon which multiple arrays are synthesized, wherein at least one array includes one or more depurination probes and/or features.

In another embodiment, a relative comparison of the detected first tag region to the detected second tag region may be used to give a proportionate amount of depurination products to yield of full length probes. The determined amount of depurination side reaction products (first tag region) can then be used to assess or evaluate the extent or magnitude of depurination that occurred during synthesis of the array.

Where the amount of first tag region nucleic acids (i.e., depurination reaction products) and/or amount of second tag region nucleic acids in a given feature or array or substrate is determined by assessing or detecting the released tag regions, the assessing or detecting signal may then be employed to make an evaluation or determination of the extent of depurination that occurred during in situ fabrication of the array. These evaluations may be performed using any convenient protocol that is capable of using signal data from one or more depurination features of the array, where the signal data may be raw or processed, to determine the magnitude of depurination.

The particular protocol employed to determine the magnitude of depurination and/or yield from the input signal data may vary, e.g., depending on the tag lengths, number of depurination probes and instrumentation used to detect the nucleic acid tag regions. A number of analytical methods may be used to detect and assess the tag regions, including, but not limited to HPLC, GC, Mass spectroscopy, gel electrophoresis, DNA chip, and combinations thereof. In certain embodiments, the intensity of the detected signal is employed to make a determination of the relative or absolute amount of first tag region that is released from the feature, array or substrate. Similarly, the intensity of a detected signal is employed to make a determination of the relative or absolute amount of the second tag region that is released from the feature, array or substrate. The determined amount of depurination side reaction products and determined amount of full length depurination probes synthesized can then be used to assess or evaluate the extent or magnitude of depurination and yield that occurred during synthesis of a given array or multiple arrays on a substrate.

One specific representative protocol for determining depurination magnitude and/or yield from an observed signal of a depurination probes includes providing the depurination probes in every QC array used to determine the quality of a manufacturing batch. Data analysis of the signals obtained for those depurination probes enable the calculation of apparent depurination and/or yield. In an embodiment, the apparent depurination and/or yield may be compared to values obtained in experiments where the depurination efficiency was modulated (for instance by varying the acid concentration) to estimate the relative quality of the synthesis. Alternatively, the apparent depurination can be compared to a control chart in order to estimate the statistical deviation from the controlled process performance. In general, increasing apparent depurination will be characteristic of decreased deblock reaction quality while variation in the apparent depurination yield will be characteristic of a drifting process.

As such, once the magnitude of depurination and/or yield is determined (e.g., in the form of a quantification, either relative or absolute, of the amount of full length and/or depurination side reaction products in a given feature), an evaluation or determination of the extent of depurination and/or yield that occurred during in situ synthesis of the array can then be made. In other words, the determined magnitude of depurination and/or yield can be employed to determine the extent of depurination and/or yield that occurred during synthesis of the array.

The determined magnitude of depurination and/or yield and therefore extent of depurination and/or yield that occurred during the in situ fabrication protocol can be employed as a quality control measure. In further embodiments, magnitude of depurination and/or yield are used as a depurination quality control measure of the amount of depurination that occurred during synthesis of the array. In still further embodiment, the magnitude of depurination and/or yield are employed in the quality evaluation of a lot or batch of arrays produced in a given in situ synthesis run, where the run includes the array displaying the depurination probes. In some of such applications, the determined magnitude of depurination is compared to a threshold depurination value, where if the determined depurination magnitude does not exceed the threshold value, the array and protocol used to prepare the same, as well as other array members of the lot or batch yield, are determined as acceptable, at least with respect to the level of depurination produced by the protocol in the member arrays of the lot or batch. Alternatively, if the determined depurination magnitude exceeds a particular threshold depurination value, then the array and protocol used to the prepare the same, as well as other array members of the lot or batch, are determined as unacceptable, at least with respect to the level of depurination produced by the protocol in the member arrays of the lot or batch. In certain embodiments, the depurination threshold can be expressed as the probability of depurination at any given A base on any given cycle. Under these circumstances, the threshold against which the determined value is compared ranges from about 0.3% to about 0.8%, such as from about 0.4% to about 0.6%.

The various embodiments described above are provided by way of illustration only and should not be construed to limit the invention. Those skilled in the art will readily recognize various modifications and changes that may be made to the present invention without following the example embodiments and applications illustrated and described herein, and without departing from the true spirit and scope of the present invention without following the example embodiments and applications illustrated and described herein, and without departing from the true spirit and scope of the present invention, which is set forth in the following claims.

The claimed invention is:

1. A method of detecting the presence of depurination reaction products on a surface of an in situ produced nucleic acid array, the method comprising:
(a) treating an in situ produced nucleic acid array with a basic solution, wherein the array includes at least one depurination probe comprising:
a first cleavage site adjacent a first tag region, wherein the first cleavage site is base-sensitive if depurinated; and
a second cleavage site adjacent a second tag region, where the second cleavage site is base-sensitive; and
(b) detecting the release of the first tag region from the array wherein detecting the release of the first tag region indicates depurination of reaction products on the array.

2. The method according to claim 1 additionally comprising:
detecting the amount of the second tag region released from the array wherein detecting the amount of the second tag region released from the array indicates synthesis yield of on the array.

3. The method according to claim 1, wherein the array includes a plurality of depurination probes and the amount of the first tag region is quantified.

4. The method according to claim 3, wherein the array includes a plurality of depurination probes and the amount of the second tag region is quantified.

5. The method according to claim 1, wherein the first tag region has a first size and the second tag region has a second size, wherein the first size and the second size are differentially detectable.

6. The method according to claim 5, wherein difference in size between the first tag region and the second tag region is at least four nucleotides.

7. The method according to claim 1, wherein the basic solution comprises an aqueous or ethanolic solution.

8. The method according to claim 1, wherein the method further comprises evaluating the level of depurination that occurred during in situ synthesis of the array.

9. The method according to claim 8, wherein the method is a method of evaluating the quality of an in situ nucleic acid array synthesis fabrication protocol.

10. The method according to claim 8, wherein the method is employed to evaluate the quality of a plurality of nucleic acid arrays fabricated on a single substrate.

11. An array having a surface, the array comprising at least one nucleic acid depurination probe attached to the surface, wherein the depurination probe comprises:
a first cleavage site proximal to the array surface and coupled to a first tag region, wherein the first cleavage site comprises one or more adenosine residues and is base-sensitive if depurinated;
a base-sensitive second cleavage site coupled to the first tag region and coupled to a second tag region;
wherein the first tag region and the second tag region are independently quantifiable following base-mediated cleavage from the array surface.

12. The array according to claim 11, wherein the first tag region and the second tag region each comprise a poly pyrimidine domains of different length.

13. The array according to claim 12, wherein the poly pyrimidine domains comprise poly-T domains ranging from about 1 to about 35 nt in length.

14. The array according to claim 11, wherein the second cleavage site comprises Thymidine-succinyl hexamide CED phosphoramidite.

15. The array according to claim 11, wherein the first cleavage site consists of two adenosine residues.

16. A nucleic acid depurination probe comprising 3′-AA-[first tag region]-[base-sensitive cleavage site]-[second tag region]-5′.

17. The nucleic acid depurination probe of claim 16, wherein the base-sensitive cleavage site comprises Thymidine-succinyl hexamide CED phosphoramidite.

18. A method of evaluating the occurrence of depurination during an in situ synthesis of a nucleic acid array comprising:
in situ synthesizing of a nucleic acid array wherein the array comprises one or more depurination probes, each probe comprising:
a first cleavage site adjacent a first tag region, wherein the first cleavage site is base-sensitive if depurinated; and
a second cleavage site adjacent a second tag region, where the second cleavage site is base-sensitive; and
deprotecting the nucleic acid array with a basic solution; and
detecting one or more first tag regions cleaved from the array wherein cleavage of a first cleavage site releasing a first tag region indicates depurination of a depurination probe.

19. The method of claim 18, wherein the first cleavage site comprises one to three adenosines and the first and second tag regions are each a poly-pyrimidine regions of different length.

20. The method of claim 18, additionally comprising detecting second tag regions cleaved from the array at the second cleavage site, wherein quantification of second tag regions indicates yield of full length depurination probes.
